(19) United States
(12) Patent Application Publication
(10) Pub. No.: US 2004/0092515 A1
(43) Pub. Date: May 13, 2004

(54) Diaminoquinazoline esters for use as dihydrofolate reductase inhibitors

(76) Inventors: Torbjjorn Lundstedt, Uppsala (SE); Anneli Hallgren, Huddinge (SE); Per Andersson, Sollentuna (SE); Anders Hallberg, Uppsala (SE); Malin Graffner Nordberg, Uppsala (SE)

Correspondence Address: Bacon & Thomas, 4th Floor, 625 Slater Lane, Alexandria, VA, 22314-1176, US

(21) Appl. No.: 10/468,151

(22) PCT Filed: Feb. 28, 2002
(86) PCT No.: PCT/GB02/00888

(30) Foreign Application Priority Data
Feb. 28, 2001 (SE) 0100694-9
Jan. 2, 2002 (SE) 0200008-1

(51) Int. Cl. A61K 31/5415; A61K 31/5377; A61K 31/517; C07D 417/02; C07D 413/02; C 07D 4 3/02

(52) U.S. Cl. 514/225.2; 514/234.2; 514/263.21; 514/252.17; 514/266.2; 514/266.21; 514/266.23; 514/266.24; 544/42; 544/116; 544/284; 544/277

(57) ABSTRACT

Compounds of the formula I:

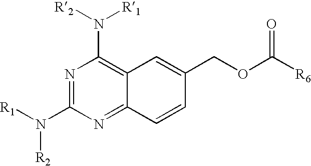

wherein

R₁, R₂, R₁′ and R₂′ are independently hydrogen or a group releasing the free amine in vivo, R₆ is a substituted phenyl or an optionally substituted, bicyclic or tricyclic aryl ring system or an optionally substituted mono, bi- or tricyclic heteroaryl ring system having utility as DHFR inhibitors with favourable pharmacokinetic properties.

### DESCRIPTION

1. Field of the Invention

This invention relates to diaminoquinazoline compounds, more particularly diaminoquinazolinyl esters, processes for preparing them, compositions containing them and uses thereof. This invention further relates to dihydrofolate reductase (DHFR) inhibitors showing improved selectivity relative to cellular reductases and/or improved pharmacokinetic profiles. The invention provides novel inhibitor compounds and methods employing such compounds for the treatment of diseases which can be therapeutically treated by immuno-modulating or cytostatic compounds, via topical, oral or parenteral means, or for the treatment of cancer forms sensitive to methotrexate. The compounds in the present invention can also be used for treating diseases/conditions involving one or several of the melanocortin receptors. Another use of these compounds involves the treatment of nephritis, e.g. IgA nephritis. Other diseases to be treated are inflammatory bowel disease i.e. ulcerative colitis and Crohn's disease, colorectal cancer, asthma, psoriasis, Pneumocystis carinii pneumonia (PCP), other serious pulmonary diseases, rheumatoid arthritis, other inflammatory conditions, other fungal infections (vaginal and others), bacterial inflammations, protozoal inflammations, cancer of the urinary bladder, cancer of the lung, other cancer types that may be reached from the “outside” of the body, non-surgical abortions (intrauterin administration) conditions associated with liver transplantation, especially in immuno-compromised individuals. The invention also provides methods of the preparation of such compounds and novel intermediates thereof.

2. Background to the Invention

Inflammatory bowel disease (IBD) is a general term that includes both ulcerative colitis and Crohn's disease, disorders of unknown etiology that result in inflammation in the gastrointestinal tract. Ulcerative colitis is an inflammatory disease of the large intestine. Ulcers develop in the inner lining, or mucosa, of the colon or rectum, often resulting in diarrhea, blood, and pus. Crohn's disease is an inflammation that extends into the deeper layers of the intestinal wall. It is found most often in the ileum and the first part of the large intestine, known as the ileocecal region.

Ulcerative colitis and Crohn's disease share many symptoms, although they also differ in important ways. Both are chronic diseases characterized by frequent relapses and remissions, and symptoms usually appear in young adults. The most common symptom of both ulcerative colitis and Crohn's disease is diarrhea. Constipation may develop during active flare-ups of both Crohn's disease and ulcertive colitis. Cramps can occur from intestinal contractions caused by inflammation. Fever, fatigue and loss of appetite are often present. Neurologic or psychiatric symptoms may be early signs of Crohn's disease when accompanied by gastrointestinal problems.

Drugs presently on the market cannot cure IBD, but some are effective in reducing the inflammation and accompanying symptoms in up to 80% of patients. Many such drugs are available, including corticosteroids, aspirin-like medications, and drugs that suppress the immune system. The primary goal of drug therapy is to put acute flares into remission and/or prevent relapse. Mesalazine is the common name of the compound 5-aminosalicylic acid or 5-ASA, which inhibits substances in the immune system that cause inflammation. 5-ASA itself is very effective and has few side effects, but it is quickly absorbed in the upper gastrointestinal tract before it can reach the colon. Sulfasalazine (Azulfidine, Salazopyrin) has been the standard 5-ASA, preparation for years. In ulcerative colitis, sulfasalazine is useful for treating mild to moderate attacks and for maintaining remission. Sulfasalazine combines 5-ASA with sulfapyridine, a sulfa antibiotic that prevents 5-ASA from being absorbed until it reaches the colon. There, intestinal bacteria break sulfasalazine into its two components. The active component, 5-ASA, blocks the inflammatory process; the other component, sulfapyridine, however, plays no positive role in the colon, and, in fact, its sulfa properties are responsible for most of the adverse side effects and allergic responses experienced by up to 30% of patients taking this drug. Some common side effects include heartburn, headache, loss of appetite, abdominal discomfort, dizziness, anemia, fever, and rashes. Sulfasalazine can also cause folic acid deficiency, and patients should take supplements of this important B vitamin.

Oral 5-ASA is used for treating active attacks of mild to moderate IBD. Olsalazine (Dipentum) is similar to sulfasalazine, in that the drug stays intact until it reaches, the intestine and is then broken down by intestinal bacteria into two components, one of which is pre-5-ASA.

Adrenal corticosteroids are powerful anti-inflammatory drugs, usually used only for active ulcerative colitis and Crohn's disease. Corticosteroids are sometimes combined with other drugs to produce more rapid symptom relief and to allow sooner withdrawals from the steroids.

For very active IBD that does not respond to standard treatments, immunosuppressant drugs are now being used for long-term treatment. All of these drugs suppress actions of the immune system and thereby its inflammatory response that causes ulcerative colitis and Crohn's disease. The two most common immuno-suppressants used for IBD are azathioprine and mercaptopurine. Other immunosuppressants being investigated for IBD and showing promising result in promoting remission include cyclosporine and methotrexate.

Metronidazole is an antibiotic used for infections caused by anaerobic bacteria and is useful for people with Crohn's disease. Other antibiotics used for Crohn's disease include trimethoprim/sulfamethoxazole, ciprofloxacin, and tetracycline.

A genetically engineered antibody shows some promising results acting against the tumor necrosis factor (TNF), a major factor in the inflammatory process that causes IBD. Recent trials are showing promising results in reducing disease activity and improving symptoms in both Crohn's disease and ulcerative colitis. A similar drug, cA2, is also showing promising result against Crohn's disease.

Asthma is a chronic lung disease and causes breathing problems. Asthma medicines keep the air tubes in the lungs open. There are two groups of asthma medicines: bronchodilators and anti-inflammatory active agents. Inhaled corticosteroids are important in therapy.

Chronic obstructive pulmonary disease (COPD) is defined as obstruction of the airways of the lungs of a persistent non-reversible nature. It is a generic term that includes chronic obstructive bronchitis, emphysema, and asthmatic bronchitis.

While advances have been made in treating of COPD, there are no quick cures and response to therapy is often marginal, with indications that those individuals who have an advanced stage of the disease have marginal chances for survival. With this said, there are indications that new procedures are on the horizon that would make this outcome not as deadly as it is now. These include new bronchodilators and anti-inflammatory agents as well as lung transplantation and lung reduction surgery.

The current standard for treating COPD is Atrovent which is a drug that has to be taken 4 times daily.

Psoriasis is a common condition affecting the skin. It causes red, scaly patches. In addition it can affect the joints, nails and eyes. Although the exact cause is unknown, psoriasis is believed to be related to faulty signals sent by the body's immune system. It has a genetic component that makes certain people more likely to develop it.

Treatments include: Moisturising creams and ointments, oils for the bath, creams, ointments, lotions and shampoos based on tar, vitamin D, salicylic acid, sun shine, stronger medications, e.g. methotrexate, and mild steroid creams and ointments, used for short periods, for psoriasis affecting the face or body folds.

The AIDS epidemic, cancer chemotherapy and organ transplantation have significantly increased the number of patients with impaired immune systems who are suffering from severe opportunistic infections including pneumonia caused by the fungus Pneumocystis carinii. P. carinii pneumonia (PCP) is a serious disease with high prevalence and constitutes the major cause of death in patients with AIDS. Current treatment of the disease with trimethoprim (TMP, structure below), a nonclassical inhibitor of dihydrofolate reductase (DHFR), in combination with a sulfonamide is still the standard therapy for PCP. Severe side-effects associated with sulfa drugs often lead to discontinuation of therapy. Inhaled aerosolised/nebulised pentamidine is used for prophylaxis. When applied systematically pentamidine exhibits a considerable toxicity. Trimetrexate (TMQ) and piritrexim (PTX), two new lipophilic agents originally developed against cancer are now used in clinic as second-line therapy (structures below). Although TMQ and PTX are both potent inhibitors of DHFR from P. carinii, they are not selective and inhibit the mammalian enzyme even more efficiently. The clinical use of TMQ and PTX is therefore limited due to their systemic host toxicity and require an expensive co-therapy with the rescue agent leucovorin (5-formyl-tetrahydrofolate). Leucovorin, a classical folate cofactor for one-carbon metabolism, is taken up via active transport only by mammalian cells and thereby reverses toxicity associated with the lipophilic DHFR inhibitors. Today considerable research efforts are devoted to the identification of more selective and potent DHFR inhibitors with the overall goal to improve therapy and to minimise the adverse effects.

Rheumatoid arthritis is another inflammatory condition, the signs and symptoms of which include: pain and swelling in the smaller joints of your hands and feet, overall aching or stiffness of the joints and muscles, especially after sleep or after periods of rest, loss of motion of the affected joints, loss of strength in muscles attached to the affected joints, fatigue, which can be severe during a flare-up, low-grade fever, deformity of the joints as time goes on.

Treatment with Nonsteroidal anti-inflammatory drugs (NSAIDs) for rheumatoid arthritis can relieve its symptoms and treatment with corticosteroids and other Disease-modifying antirheumatic drugs (DMARDSs) can slow or halt its progression. NSAIDs can, however, lead to such side effects as indigestion and stomach bleeding, as well as damage to the liver and kidneys, ringing in the ears (tinnitus), fluid retention, and high blood pressure. COX-2 inhibitors, a new class of NSAIDs, may be less damaging to the stomach, but may have higher incidents of other side-effects than conventional NSAIDs. Corticosteroids reduce inflammation and slow joint damage. DMARDs are another group of drugs prescribed. Some commonly used DMARDs include hydroxychloroquine sulfate (Plaquenil), gold compounds (Ridaura, Solganal), sulfasalazine (Azulfidine) and minocycline (Minocin). Other forms of DMARDs include immunosuppressants and TNF blockers. Some of the commonly used immunosuppressants include methotrexate, leflunomide, azathioprine, cyclosporine and cyclophosphamide.

These medications can have potentially serious side effects such as increased susceptibility to infection and disease.

Antifolate compounds are used in the treatment of bacterial infections. Sulfonamides are structural analogues of p-aminobenzoic acid. They interfere with the early stages of folic acid synthesis by competitive inhibition of dihydropteroic acid synthetase, which condenses p-aminobenzoic acid with dihydropteroic acid. The sulfonamide may also be erroneously incorporated into the folic acid molecule to produce an inactive product. Bacterial cells synthesize folic acid, whereas mammalian cells use the preformed dietary vitamin, and this is the basis of the selective antibacterial action of sulfonamides.

Diaminopyrimidines, like trimethoprim and the antimalarial compound, pyrimethamine, act at a later stage on the same pathway by inhibiting dihydrofolate reductase, the enzyme that generates the active product, tetrahydrofolate, from dihydrofolate. The affinity of trimethoprim for DHFR of bacteria is several orders of magnitude higher than the affinity for the mammalian enzyme; similarly pyrimethamine has a very high affinity for the DHFR of malaria parasites.

Because sulfonamides and diaminopyrimidines act on the same metabolic pathway, they exhibit a strongly synergic interaction, at least in vitro. However, because tetrahydrofolate is reoxidized to dihydrofolate during the biosynthesis of thymidylic acid, diaminopyrimidines rapidly trap the vitamin in the unusable dihydrofolate form. Sulfonamides, in contrast, cut off the supply of dihydrofolate and act rather slowly because the folate pool becomes depleted only after several cell divisions. For this reason, if there is sufficient diaminopyrimidine present to halt tetrahydrofolate regeneration completely, the sulfonamide does not have an opportunity to contribute to the antibacterial action.

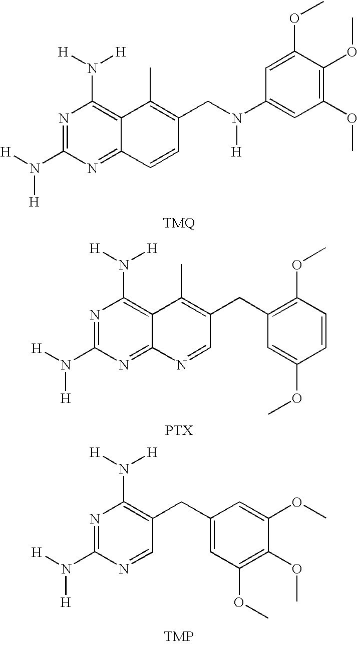

Certain ester analogues of the above DHFR inhibitors have been published in the academic literature. In particular Hallberg et al. (Chem. Pharm. Bull., 1998, 46, 591-601 and J. Med. Chem., 2000, 43, 3852-3861) describes compounds of the formula:

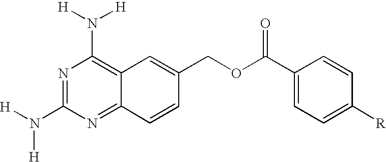

wherein R is hydrogen or a glutamic acid linked to the phenyl ring by an amide bond. However, the R=hydrogen variant showed a poor hydrolysis rate in in vitro assays comprising pig liver esterase, cholesterol esterase, human duodenal mucosa, human liver, rat liver and human leukocytes, respectively, leading the authors to conclude that “esters comprising a 2,4-diamino-pyrimidine ring are not suitable as soft drugs” (Chem. Pharm. Bull., 1998, 46, 591-601). The glutamic acid derivative exhibited a pharmacokinetic profile similar to methotrexate and was as expected relative inert to ester hydrolysis in vivo in rat (J. Med. Chem., 2000, 43, 3852-3861).

### BRIEF DESCRIPTION OF THE INVENTION

We have now discovered that certain active isostenic-isoelectronic analogues of lipophilic DHFR structures tend to be deactivated by a fast hydrolytic metabolism in vivo. These DHFR inhibitors, consisting of an ester in the middle region, are thus more easily metabolised than the corresponding non-lipophilic ester analogues of classic DHFR inhibitors, and will in general be administered near the site of action according to the criteria for the soft drug concept (Med. Res. Rev., 2000, 20, 58-101). The invention thus provides a new entry to efficient and safe treatment of diseases which can be therapeutically treated by immuno-modulating or cytostatic effective compounds, in particular in the form of dihydrofolate reductase inhibitors, either applied topically, orally or parenterally, or cancer forms sensitive to methotrexate. IBD, i.e. ulcerative colitis and Chron's disease, is a further indication that can be treated, and some others are colorectal cancer, cancer of the urinary bladder, the lung and other cancer types that may be reached from the “outside” of the body, psoriasis, PCP, other fungal (vaginal and others), protozoal and bacterial (pulmonary infections, urinary tract infections and others) infections, non-surgical abortions (intrauterin administration), asthma, or other serious pulmonary diseases, rheumatoid arthritis and other inflammatory conditions. Other applications are as agents for non-rejecting liver transplantation, and intestine transplantation. As a short-lived duration of exposure is sufficient, systemic treatment of e.g., rheumatoid arthritis or other inflammatory conditions, is possible as well. The compounds of the present invention can also be used for treatment of nephritis, e.g. IgA nephritis, which is an inflammatory-like condition in the kidneys. The compounds in the present invention can also be used for treating diseases/conditions involving one or several of the melanocortin receptors.

The invention is as defined in the claims.

More particularly, the invention provides novel compounds of formula I:

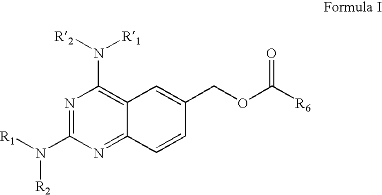

wherein R₁, R₂, R₁′ and R₂′ are hydrogen.

R₆ is a substituted phenyl group, optionally substituted heteroaromatic group or optionally substituted aromatic group.

As regards optionally substituted heteroaromatic groups, R₆ may be an optionally substituted mono-, bi- or tricyclic ring system comprising 1-4 heteroatoms (preferred monocyclic rings being furyl, thienyl, pyrrolyl, pyrazolyl, imidazolyl pyridyl, pyrazinyl, pyrimidinyl, pyridazinyl, oxazolyl, isoxazolyl, thiazolyl, isothiazolyl and the like; preferred bicyclic rings being one of the above-mentioned monocylic rings fused to a phenyl ring such as quinolinyl, isoquinolinyl, benzimidazolyl, benzothiazolyl, benzoxazolyl, benzothienyl, indolyl, isoquinolinyl and the like, or phenyl fused to an unsaturated or partially saturated ring such as pyranyl, pyrrolinyl, pyrrolidinyl, pyrazinyl, piperazinyl, oxazolidinyl, isooxazidinyl, morpholinyl, thiazolidinyl, and the like; and preferred tricyclic rings being acridine, xanthene, thioxanthene, phenthiazine, thianthrene, dibenzazepine, carbazol, and the like). The heterocyclic ring system may be bonded via a carbon or via a hetero atom and may be bonded from a hetero atom containing ring or a carbocyclic ring.

Alternatively R₆ may be a substituted phenyl, or optionally substituted bicyclic or tricyclic carbocyclic, aromatic ring system (preferred systems being indanyl, naphthyl, diphenylmethyl, fluorene, anthracene, phenanthrene and the like).

The optional substituents on R₆ include halo, C₁-C₆ alkyl, C₁-C₆ alkoxy, C₁-C₆ alkenyl, C₁-C₆ alkynyl, C₁-C₆ alkanoyl, thioC₁-C₆ alkyl, haloC₁-C₆ alkyl, phenyl, benzyl, furyl, thienyl, pyrazolyl, oxazolyl, isoxazolyl, pyrrolyl, thiazolyl, isothiazolyl, NHC₁-C₆ alkyl, N(C₁-C₆ alkyl)₂, thioC₁-C₆alkoxy, hydroxyC₁-C₆alkyl, aminoC₁-C₆alkyl, cyano, carbalkoxy, benzyloxy, morpholyl-C₁-C₆ alkyloxy, a monocyclic carbo- or heterocycle, as defined above, or a carbo- or heterocyclic group spaced by alkyl, such as C₁-₃ alkylaryl.

R₆ is preferably selected from:

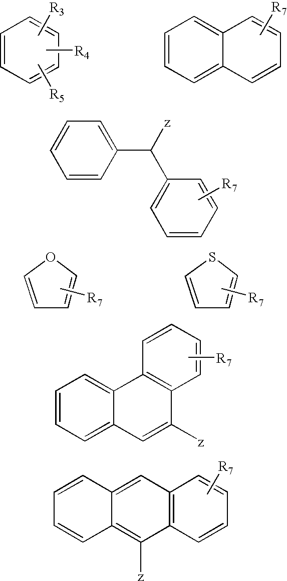

wherein z denotes the point of attachment.

R₇ may be hydrogen, halo, C₁-C₆ alkyl, C₁-C₆ alkoxy, C₁-C₆ alkenyl, C₁-C₆ alkynyl, C₁-C₆ alkanoyl, thioC₁-C₆ alkyl, haloC₁-C₆ alkyl, phenyl, benzyl, furyl, thienyl, pyrazolyl, oxazolyl, isoxazolyl, pyrrolyl, thiazolyl, isothiazolyl, NHC₁-C₆ alkyl, N(C₁-C₆ alkyl)₂, thioC₁-C₆alkoxy, hydroxyC₁-C₆alkyl, aminoC₁-C₆alkyl, cyano, carbalkoxy, benzyloxy, morpholyl-C₁-C₆ alkyloxy, a monocyclic carbo- or heterocycle, as defined above, or a carbo- or heterocyclic group spaced by alkyl, such as C₁₋₃ alkylaryl.

Favoured lipophilic substituents as R₇ include C₁-C₆ alkyl, C₁-C₆ alkoxy, halo, especially fluoro or bromo, phenyl, benzyl, thienyl, haloC₁-C₆alkyl, thioC₁-C₆ alkyl.

R₃ may be halo, C₁-C₆ alkyl, C₁-C₆ alkoxy, C₁-C₆ alkenyl, C₁-C₆ alkynyl, C₁-C₆ alkanoyl, thioC₁-C₆ alkyl, haloC₁-C₆ alkyl, phenyl, benzyl, furyl, thienyl, pyrazolyl, oxazolyl, isoxazolyl, pyrrolyl, thiazolyl, or isothiazolyl.

The term halo herein includes chloro, bromo, fluoro and iodo.

R₄ and R₅ may each be either hydrogen or a substituent as defined for R₃.

Favoured lipophilic substituents for R₃, and optionally, R₄ & R₅ include C₁-C₆ alkyl, (for example C₁-C₃ alkyl, such as methyl, ethyl, n- or i-propyl), C₁-C₆ alkoxy (for example C₁-C₃ alkoxy such as methoxy), thioC₁-C₆ alkyl, for example C₁-C₃ thioalkyl, (such as thiomethyl or thioethyl), haloC₁-C₆ alkyl (for example haloC₁ such as —CF₃), phenyl, thienyl, or benzyl, any of which cyclic substituents being optionally substituted as defined herein for other substituents. Exemplary substituents for R₃-R₅ include 3,4,5-trimethoxy, 2,5-dimethoxy, halo, or 3,5-dimethyl.

Some preferred alkoxy groups are methoxy, ethoxy, n-propoxy, isopropoxy, n-butoxy, sec.-butoxy, tert.-butoxy, n-pentoxy and n-hexoxy.

Groups for R₁, R₂, R₁′ and R₂′ of formula I which release the amine in vivo include conventional pharmaceutically acceptable amide prodrugs such as haloC₁-C₃ alkyl, C₁-C₃ alkyl, such as cyclopropyl or those described in U.S. Pat. No. 4,760,057, U.S. Pat. No. 5,466,811 or WO 90/08128.

Although certain of the novel compounds may show decreased potency against pathogen or cellular DHFR relative to current inhibitors, they may exhibit somewhat better selectivity versus liver or other key DHFR species and, importantly, a more rapid metabolism to inactive metabolites in vivo.

Accordingly, the compounds of the invention have utility in the treatment of diseases which can be therapeutically treated by immuno-modulating or cytostatic compounds, either applied topically, orally, rectally, or parenterally, or cancer forms being sensitive to methotrexate. Another utility are the treatments of IBD, i.e. ulcerative colitis and Crohn's disease. asthma, PCP, psoriasis, rheumatoid arthritis, other inflammatory conditions, colorectal cancer, cancer of the urinary bladder, cancer of the lung and other cancer types that may be reached from the “outside” of the body, inflammatory conditions caused by bacterial, fungal (vaginal and others) or protozoal infections, non-surgical abortions (intrauterin administration), liver transplantation, other serious pulmonary, especially in immuno-compromised individuals.

A further aspect of the invention thus provides the compounds of formula I for use in therapy, such as use in the manufacture of a medicament for the treatment of disorders requiring the inhibition of DHFR.

The activities of compounds of the invention against various cellular and pathogen DHFR are measured by conventional assays, such as those described in Antimicrob. Agents Chemother., 1991, 35, 1348-1355 and Antimicrob. Agents Chemother., 1993, 37, 1914-1923. Preferred compounds will typically show a low IC50 for the target DHFR, and under certain circumstances in the matter of treating P. carinii, show, in conjunction with high selectivity index between cellular DHFR (for example rat liver DHFR) and the relevant pathogen DHFR. As a reference point it should be noted that conventional DHFR inhibitors such as PTX and TMQ have selectivity indexes of the order 0.13-0.19, which is generally exceeded in the compounds of the invention.

The compounds of formula I can form salts, which form an additional aspect of the invention. Appropriate pharmaceutically acceptable salts of the compounds of formula I include salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, lactobionate, pivolate, camphorate, undecanoate and succinate, organic sulphonic acids such as methanesulphonate, ethanesulphonate, 2-hydroxyethane sulphonate, camphorsulphonate, 2-napthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Hydrochloric acid salts are convenient.

The compounds of formula I may be isolated as the hydrate or as the free base. The compounds of the invention may be isolated in crystal form, preferably homogenous crystals, and thus an additional aspect of the invention provides the compounds of formula I in substantially pure crystalline form, comprising >70%, preferably >90% homogeneous crystalline material for example >95% homogeneous crystalline material, more preferably >99% homogenous material.

The compounds of the invention are particularly suited to topical administration, such as pulmonary, dermally, optically, vaginally, nasally, transdermally but may also be administered orally, rectally, or parenterally, for instance orally in a bioadhesive composition to adhere to the gastro-intestinal tract or parenterally as intramuscularly, intrapernitoneally, intravenously or epidurally. The compounds may be administered alone, for instance in a capsule, but will generally be administered in conjunction with a pharmaceutically acceptable carrier or diluent. The invention extends to methods for preparing a pharmaceutical composition including a compound of Formula I or its pharmaceutically acceptable salt in conjunction or association with a pharmaceutically acceptable carrier or vehicle.

The term topical herein means any application on the outside of the body but also applies to the topical administration on the mucous membranes of the gastro-intestinal tract, such as by means of a mucoadhesive composition adhering to e.g., the intestines where it serves its therapeutic effect.

Oral formulations are conveniently prepared in unit dosage form, such as capsules or tablets, employing conventional carriers or binders such as magnesium stearate, chalk, starch, lactose, wax, gum or gelatine. Liposomes or synthetic or natural polymers such as HPMC or PVP may be used to afford a sustained release formulation. Alternatively the formulation may be presented as a nasal or eye drop, syrup, gel or cream comprising a solution, suspension, emulsion, oil-in-water or water-in-oil preparation in conventional vehicles such as water, saline, ethanol, vegetable oil or glycerine, optionally with flavouring agent and/or preservative and/or emulsifier. Any formulation may contain 0.5 to 99.5% by weight of the therapeutically active compound.

### METHODS OF PREPARATION

The following examples are intended to illustrate but not to limit the scope of the invention, although the compounds named are of particular interest for the intended purposes. The preparation of the compounds of general formula (I) is presented schematically in examples below. The compounds are numbered and listed with their complete name below.

The compounds having the general formula (I) may be prepared by the following general methods.

### Method 1

A carboxylic acid of formula (II), protected or activated as necessary, wherein R₆ is as previously defined, is reacted with a compound of formula (III), protected or activated as necessary, wherein L is a suitable leaving group such as halogen and alkyl- or arylsulfonate and R₁, R₂, R₁′ and R₂′ are as previously defined, by using standard esterification procedures well known in the art, and a compound of Formula (I) is obtained after deprotection where necessary or desired.

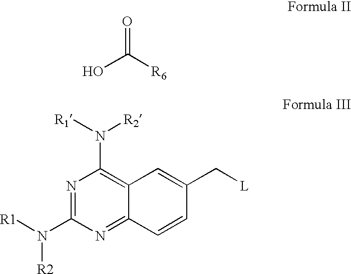

### Method 2

A compound of formula (IV), protected or activated as necessary, wherein R₁, R₂, R₁′ and R₂′ are as previously defined, is reacted with a compound of formula (V), protected or activated as necessary, wherein R₆ is as previously defined and wherein L is a suitable leaving group such as halogen and alkyl- or arylsulfonate, by using standard esterification procedures well known in the art, and a compound of Formula (I) is obtained after deprotection where necessary or desired.

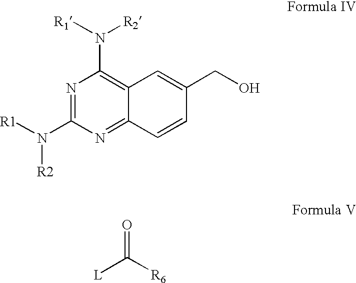

### DETAILED SYNTHETIC DESCRIPTION

Scheme 1 outlines the synthetic approach employed for the preparation of some exemplary target compounds (encompassing an ester bond in the bridge between the aromatic ring systems) listed in the table at the foot of Scheme 1. The synthesis of compounds 15a, 1a and 2a, as well as the metabolites 18a and 19a is described in Chem. Pharm. Bull., 1998, 46,591-601. Thus, protection of the amino groups of 16a with pivalic anhydride in anhydrous DMF afforded the dipivaloated aldehyde 17a, which was further oxidised to the corresponding carboxylic acid with sodium chlorite, employing 2-methyl-2-butene as a scavenger, J. Org. Chem. 1980, 45, 1175-1176, Acta Chem. Scand. 1973, 27, 888-890. After subsequent esterification, the quinazoline esters were depivaloated using ammonia in dioxane, Heterocycles 1993, 36, 1883-1895, finally yielding the desired esters 1a and 2a. Treatment with Ni—Al alloy in refluxing formic acid, Chem. Pharm. Bull. (Tokyo) 1998, 46, 591-601, converted the nitrile 15a into the aldehyde 16a, which was further reduced to the corresponding alcohol 19a using sodium borohydride in methanol. Too long reaction-time lead to over-reduction of the alcohol to the 6-methyl analogue, J. Heterocycl. Chem. 1990, 27, 1-12. The bromination of the alcohol 19a was performed with methods employed in antifolate chemistry, including the use of dibromotriphenylphosphorane (Ph₃PBr₂) in DMAc, J. Org. Chem. 1977, 42, 208-211, HBr in dioxane J. Med. Chem. 1986,709-715, J. Med. Chem. 1992, 35, 332-337, PBr₃ in THF, J. Org. Chem. 1981, 46, 1777 1781, or HBr in AcOH, J. Med. Chem. 1993, 36, 4161-4171, respectively. The last procedure provided the best results in the present systems. Displacement of the bromide with the appropriate carboxylic acids using potassium carbonate or cesium carbonate as bases in DMF, DMSO or DMAc, respectively, afforded the desired esters in various yields, Scheme 1.

Scheme 1

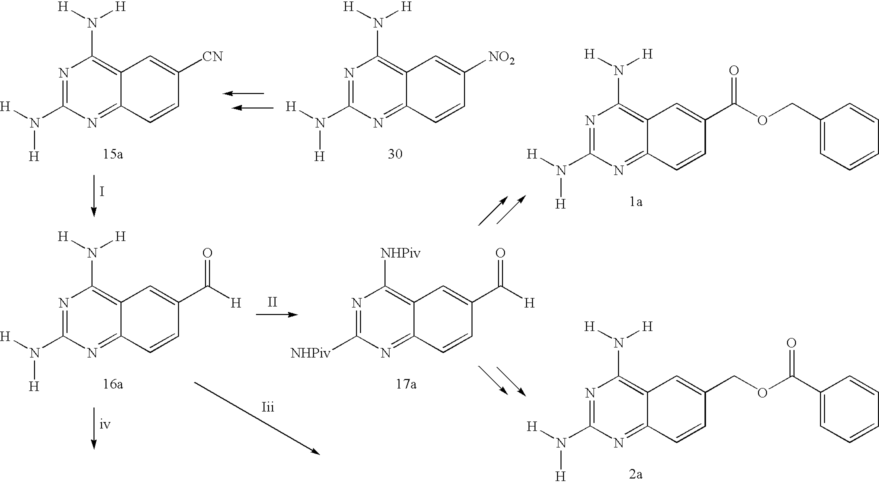

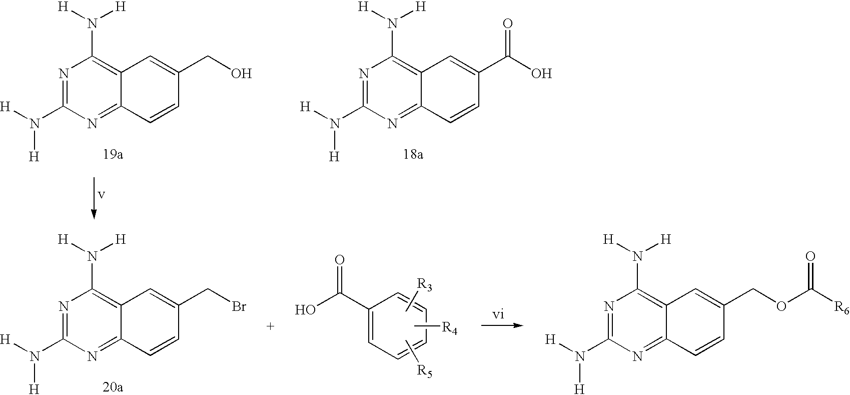

| Compound | R₆ |
|---|---|
| 3a | 3,4,5-OCH₃-phenyl |
| 4a | 2,5-OCH₃-phenyl |
| 5a | 2,3,4-OCH₃-phenyl |
| 6a | 3,5-OCH₃-phenyl |
| 7a | 3.4-OCH₃-phenyl |
| 101 | 1-naphthyl |
| 102 | 2-naphthyl |
| 103 | diphenylmethyl |
| 104 | 2-thienyl |
| 105 | 3-thienyl |
| 106 | 2-furyl |
| 107 | 3-furyl |
| 108 | 2-biphenyl |
| 109 | 4-biphenyl |
| 110 | 2-benzylphenyl |
| 111 | 3,5-dimethylphenyl |
| 112 | 9-phenanthrene |
| 113 | 9-anthracene |
| 114 | 3-iodophenyl |

### Chemistry. General Comments

¹H- and 13C-NMR spectra were recorded on a JEOL JNM-EX270 spectrometer at 270 and 67.8 MHz, respectively and a JEOL JNM-EX400 spectrometer at 400 and 100 MHz, respectively. Thin-layer chromatography (TLC) was performed by using aluminium sheets precoated with silica gel 60 F254 (0.2 mm) type E; Merck. Chromatographic spots were visualized by UV light, or by an acidic ethanolic solution of 2,4-dinitrophenylhydrazine. Column chromatography was conducted on silica gel S (0.032-0.063 mm; Riedel-de Haën®), silica gel 60 (0.040-0.063 mm; E. Merck), unless otherwise noted. Preparative TLC was performed on glass sheets precoated with silica gel 60 F₂₅₄ (2.0 mm; E. Merck). Centrifugal chromatography was carried out on a Harrison Research Chromatotron (model 7924T) with silica gel PF₂₅₄ containing gypsum (E. Merck) as solid phase. Melting points (uncorrected) were determined in open glass capillaries on an Electrothermal apparatus. Infrared (M) spectra were recorded on a Perkin-Elmer 1605 FT-IR spectrophotometer and are recorded in νₘₐₓ (cm⁻¹).

The Quest 210 organic syntheziser (Argonaut Technologies) was used in the syntheses of 105-106, 108-113. In the syntheses of 115 and 116 microwave heating was performed in a Smith Synthesizer™ single mode microwave cavity producing continuous irradiation at 2450 MHz (Personal Chemistry AB, Uppsala, Sweden). The syntheses were performed in heavy-walled glass Smith Process Vials sealed with aluminum crimp caps fitted with a silicon septum. The inner diameter of the vial filled to the height of 2 cm was 1.3 cm. Reaction mixtures were stirred with a magnetic stir bar during the irradiation. The temperature, pressure and irradiation power were monitored during the course of the reaction. The average pressure during the reaction was 3-4 bar. After completed irradiation, the reaction tube was cooled with high-pressure air until the temperature had fallen below 39° C. (ca. 2 min). The microwave irradiations were performed under controlled conditions that make the procedure highly safe, reliable and reproducible. Single mode irradiation with monitoring of temperature, pressure and irradiation power vs. time was used throughout. The reaction temperature was kept constant throughout the reaction in the single mode cavity by an automatic power control. When carrying out reactions with microwave irradiation in closed vessels, extra caution is advisable. In addition to the high pressure generated by the vapour pressure of volatile components, the metal catalysts might precipitate and cause a “thermal runaway”, increasing the pressure further. Therefore, the use of special heavy-walled process vials is highly recommended. SpeedVac® Plus SC250DDA (Savant) was used in the evaporations of DMF. The elemental analyses were performed by Mikro Kemi AB, Uppsala, Sweden or Analytische Laboratorien, Gummersbach, Germany and were within ±0.4% of the calculated values. All commercial chemicals were used without further purification.

### Starting Material 2,4-Diamino-6-(bromomethyl)quinazoline (20a)

Compound 20a was prepared from 19a according to the following procedure. The alcohol (0.53 mmol) was dissolved in acetic acid (14 ml) and heated up to 60° C. when necessary, until everything was dissolved. The solution was cooled down to room temperature before 30% hydrobromic acid in acetic acid (18 ml) was poured down to the reaction mixture. After stirring at room temperature for 24-48 h, in the absence of light, the mixture was poured down with stirring to cold diethyl ether. The bromomethyl compound generated as its hydrobromic salt, was allowed to precipitate in the refrigerator for a couple of hours before it was collected under nitrogen and washed with cold diethyl ether. Compound 20a was dried in vacuo at 40° C. and used in the next step without further purification.

### 1:1

### 3,4,5-Trimethoxy-benzoic acid 2,4-diamino-quinazolin-6-ylmethyl ester (3a)

Freshly prepared 20a (1.58 mmol) in DMSO (15 ml) was added dropwise to a mixture of 3,4,5-trimethoxybenzoic acid (368 mg, 1.7 mmol) and powdered potassium carbonate (523 mg, 3.8 mmol) in anhydrous DMSO (5 ml). The reaction mixture was allowed to stir under an atmosphere of nitrogen for 48 h at room temperature, after which water (200 ml) was added and the flask was put in the refrigerator. The precipitate was collected by filtration and washed with water. A solution of the crude product in methanol containing suspended silica gel was evaporated in vacuo. The silica plug was loaded on the top of a silica column, previously conditioned with dichloromethane, and the crude ester was purified by repeated flash chromatography, using CHCl₃:Me0H (19:1) as eluent, affording 129 mg (21% over two steps) of a white powder: mp 223-227° C.; IR (KBr) 1702 (ester) cm⁻¹;⁻¹H NMR (DMF-d₆) δ 8.23 (d, J=1.70 Hz, 1H, H-5), 7.62 (dd, J=8.55, 1.92 Hz, 1H, H-7), 7.37 (br s, 2H, NH₂), 7.36 (s, 2H, ArH), 7.28 (d, J=8.55 Hz, 1H, H-8), 6.02 (br s. 2H, NH₂), 5.39 (s. 2H, CH₂), 3.96 (s, 6H), 3.82 (s, 3H); ¹³C NMR (DMSO-d₆) δ 165.28, 162.45, 161.06, 152.76, 152.56, 141.82, 132.90, 127.21, 124.76, 124.52 (2C), 123.97 (2C), 109.89, 106.55 (2C), 66.67, 60.16, 56.00.(2C). Anal. (C₁₉H₂₀N₄O₅) C, H, N.

### 1:2

### 2,5-Dimethoxy-benzoic acid 2,4-diamino-quinazolin-6-ylmethyl ester (4a)

Compound 4a was prepared from 20a (1.58 mmol) in anhydrous DMF (15 ml) which was added dropwise to a mixture of 2,5-dimethoxybenzoic acid (862 mg, 4.73 mmol) and potassium carbonate (654 mg, 4.73 mmol) in dry DMF (5 ml). After 48 h under an atmosphere of nitrogen at room temperature the mixture was filtered off and the crude product was evaporated under reduced pressure on a small portion of silica gel. The silica plug was loaded on the top of a silica column, previously conditioned with dichloromethane, and the crude ester was purified by repeated flash chromatography, using CHCl₃:MeOH (39:1) as eluent, providing 160 mg (29% over two steps) of the pure ester: mp, 195-197° C.; IR (KBr) 1703 (ester) cm^{-I.}; ¹H NMR (DMF-d₆) δ 8.22 (app s, 1H, H-5), 7.69 (dd, J=8.57, 1.98 Hz. 1H, H-7), 7.48 (br s, 2H, NH₂), 7.31 (d, J=8.58 Hz, I H, H-8), 7.29-7.17 (m, 3H, ArH), 6.15 (br s, 2H, NH₂), 5.34 (s. 2H, CH₂), 3.83 (s, 3H), 3.80 (s, M); ¹³C NMR (DMF-d₆) δ 165.89, 163.50, 161.67, 153.39, 153.28, 152.60, 133.39, 128.52, 124.84, 124.21, 121.62, 118.9 1,-116.14, 114.66, 110.51, 66.85, 56.59, 55.73. Anal. (C₁₈H₁₈N₄O₄. 0.25H₂O) C, H, N.

### 1:3

### 2,3,4-Trimethoxy-benzoic acid 2,4-diamino-quinazolin-6-ylmethyl ester (5a)

Compound 5a was prepared as described above for 4a starting with 20a (0.53 mmol), 2,3,4-trimethoxybenzoic acid (335 mg, 1.58 mmol), and potassium carbonate (218 mg, 1.58 mmol) in DMF (total amount: 5 ml). The reaction was allowed to proceed for 2 days before it was filtered and worked-up as described above for 4a yielding 34 mg (17% over two steps): ¹H NMR (DMF-d₆) δ 8.19 (app s, 1H, H-5), 7.65 (dd, J 8.57Y 1.98 Hz, 1H, H-7), 7.53 (d, J=8.91 Hz. 1H, ArH), 7.51 (br s, 2H, NHA 7.28 (d, J=8.58 Hz, 1 H, H-8), 6.91 (d, J8.91 Hz, 1 H, ArH), 6.24 (br s, 2H, NH₂), 5.28 (s, 2H, CH₂), 3.88 (s, 314), 3.78 (s, 314), 3.76 (s, M); ¹³C NMR (DMF-d₆) δ 165.71, 163.84, 161.98. 158.02, 154.90, 152.85, 143.63, 133.91. 128.97, 127.13, 125.12, 124.74, 118.69, 110.87, 108.34, 67.08, 62.05, 60.95, 56.50. Anal. (C₁₉H₂₀N₄O₅ 0.25H₂O) C, H, N.

### 1:4

### 3,5-Dimethoxy-benzoic acid 2,4diamino-quinazolin-6-ylmethyl ester (6a)

Compound 6a was prepared as described above for 4a starting with 20a (0.53 mmol), 3,5-dimethoxybenzoic acid (287 mg, 1.58 mmol), and potassium carbonate (218 mg, 1.58 mmol) in DMF (total amount: 5 ml). The reaction was allowed to proceed for 2 days before it was filtered and worked-up as for 4a, yielding 42 m-(23% over two steps): ¹H NMR (DMF-d₆) δ 8.19 (d, J=1.65 Hz, 1H, H-5), 7.63 (dd,I=8.58, 1.65 Hz, I H, H-7), 7.42 (br s, 2H, NH2), 7.25 (d, J=8.58 Hz, IH, H-8), 7.11 (d, J=2.31 Hz, 2H, ArH), 6.77 (t, J=2.31 Hz, 1 H, ArH), 6. 10 (br s, 2H, NH₂), 5.33 (s, 2H, CH₂), 3. 81 (s, 6H); ¹³C NMR (DMT-d₆) δ 166.32, 163.80, 162.92, 161.66, 153.90, 133.62, 132.79, 128.37, 125.77, 124.69, 110.98, 107.76 (2C), 105.57, 67.62, 55.98 (2C). Anal. (C₁₈H₁₈N₄O₄) C, H, N.

### 1:5

### 3,4-Dimethoxy-benzoic acid 2,4-diamino-quinazolin-6-ylmethyl ester (7a)

Compound 7a was prepared as described above for 4a starting with 20a (0.53 mmol), 3,4-dimethoxybenzoic acid (287 mg, 1.58 mmol), and potassium carbonate (218 mg, 1.58 mmol) in DMF (total amount: 5 ml). The reaction was allowed to proceed for 2 days before it was filtered and worked-up as for 4a yielding 56 mg (30% over two steps) of the pure ester: ¹H NMR (DMF-d6) δ 8.19 (d, J=1.65 Hz, 1H, H-5), 7.63 (dd, J=8.25, 1.98 Hz, 1H, H-7), 7.62 (dd, J=8.58, 1.98 Hz, 1H, H-7), 7.52 (d, J=1.98 Hz 2H, ArH), 7.44 (br s, 2H, NH₂),7.25 (d, J=8.58 Hz, 1H, H-8), 7,08 (d, J=8.25 Hz, 1 H, ArH), 6.11 (br s, 2H, NH₂), 5.31 (s, 211, CH₂), 3.87 (s, 3H, CH₃), 3.84 (s, 3H,CH₃); ¹³C NMR (DMF-d6) δ 166.41, 163.79, 162.95, 154.19, 153.82, 149.61, 133.58, 128.68, 125.71, 124.55, 124.06, 122.98, 112.60, 111.68, 110.96, 67.14, 56.22, 56.06. Anal. (C₁₈H₁₈N₄O₄) C, H, N.

### Starting Material 2,4-Diamino-6-iodoquinazoline (21a)

(Harris, N. V.; Smith, C.; Bowden, K. A Simple Synthesis of 5,8,10-Trideazamino-ptenin Analogues. Synlett 1990, 577-578, Harris, N. V.; Smith, C.; Bowden, K. Antifolate and Antibactenial Activities of 6-Substituted 2,4-Diaminoquinazolines. Eur. J. Med. Chem. 1992, 27, 7-18.) Sodium nitrite (0.207 g, 3.00 mmol) in 1 ml of water (0° C.) was added to a cold solution of 2,4-diaminoquinazoline (0.5 g, 2.9 mmol) in 2M hydrochloric acid (8 ml) and was allowed to stir on ice-bath for 15 min. Potassium iodide (0.498 g, 3.00 mmol) in water (4 ml) was added to the mixture and the reaction was allowed to proceed for an additional 2 h before it was heated up to 70° C. only for a short while. The reaction mixture was cooled down to room temperature and 7M aqueous ammonia (4 ml) was added and the precipitate was filtered off. The solid was dissolved in DMF and filtered. After precipitation with water and another filtration, the crude mixture was purified by flash chromatography [CH₂Cl₂:Me0H+NH₃ (aq) (9:1)]. Concentration of the pooled fractions gave a powder which was recrystallised from DMF and water, yielding 166 mg (20%) of pure product: ¹H NMR (DMSO-d₆) δ 8.37 (d, J=1.65 Hz, 1H, H-5), 7.72 (dd, J=8.58, 1.98 Hz, 1H, H-7), 7.45 (br s, 2H, NH₂), 6.99 (d, J 8.91 Hz, 1H, H-8), 6.24 (br s, 2H, NH₂).

The synthetic approach employed for the preparation of the target compounds 101-104, 107, and 114, respectively, was the same as described earlier for the preparations of e.g., 4a-7a, with displacement of the bromide in 2,4-diamino-6-bromomethylquinazoline (20a) with the appropriate carboxylic acids in DMF using potassium carbonate as a base. The synthesis of the esters 105-106, and 108-113 were conducted in the Quest 210 organic syntheziser. Suzuki-couplings with 2,4-diamino-6-iodoquinazoline (21a) (Harris, N. V.; Smith, G; Bowden, K. A Simple Synthesis of 5,8,10-Trideazaminopterin Analogues. Synlett 1990, 577-578, Harris, N. V.; Smith, C.; Bowden, K. Antifolate and Antibacterial Activities of 6-Substituted 2,4-Diaminoquinazolines. Eur. J. Med. Chem. 1992, 27, 7-18.) with phenylboronic acid, and 2,6-dimethoxyphenyl boronic acid, respectively, were conducted in a Smith Synthesizer™ single mode microwave cavity for the synthesis of the esters 115-116. Different conditions were run, trying out different conditions using the Smith reaction kit for Suzuki couplings. Pd(PPh₃)₂Cl₂, Pd(OAc)₂, and Hermanns catalyst (trans-di-μ-acetobis[2-(di-o-tolylphosphino)benzyl]dipalladium(II)), respectively, was used as catalysts together with sodium or cesium carbonate in solutions consisting of DME:H₂O:EtOH (7:3:2) or DMF. Full conversion to the diphenyl analogues was accomplished after 120 seconds at 140° C. using, Pd(PPh₃)₂Cl₂ and sodium carbonate in DME:H₂O:EtOH (7:3:2).

### 1:6

### (2,4-Diaminoquinazoline-6-yl)methyl 1-Naphthoate (101)

2,4-Diamino-6-bromomethylquinazoline (20a) (0.53 mmol) in anhydrous DMF (2 ml) was added dropwise to a mixture of 1-naphthoic acid (272 mg, 1.58 mmol); and potassium carbonate (218 mg, 1.58 mmol) in DMF (total amount: 5 ml). The reaction mixture was stirred under nitrogen at room temperature for 2 days before the crude product was evaporated under reduced pressure on a small portion of silica gel. The silica plug was loaded on the top of a silica column, previously packed with dichloromethane, and the crude ester was purified by repeated flash chromatography [CH₂Cl₂:Me0H+NH₃ (19:1 yielding 80 mg (44% over two steps): ¹H NMR (DMF-d6) δ 8.88-8.84 (app d, IFI, ArH), 8.26 (d, J=1.98 Hz, 1H, H-5), 8.22-8.18 (m, 2H, ArH), 8.05-8.02 (m, 1H, ArH), 7.71 (dd, J=8.58, 1.98 Hz, 111, H-7), 7.68-7.55 (m, 3H, AM), 7.46 (br s, 2H, NH₂), 7.28 (d, J=8.58 Hz, 1H, H-8), 6.12 (br s, 2H, NH₂), 5.45 (s, 2H, CH₂); ¹³C NMR (DMF-d6) δ 167.61, 163.80, 162.90, 153.92, 134.60, 134.19, 133.78, 131.71, 130.90, 129.48, 128.51, 128.44, 127.63, 127.07, 126.01, 125.76, 125.57, 124.8 1, 111.01, 67.64. Anal. (C₂₀H₁₆N₄O₂.0.4H₂O) C, H, N.

### 1:7

### (2,4-Diaminoquinazoline-6-yl)methyl 2-Naphthoate (102)

Compound 102 was prepared as described for 101 using 2-naphthoic acid (272 mg, 1.58 mmol). The reaction was allowed to proceed for 2 days before it was filtered and worked-up as described above for 101 yielding 60 mg (33% over two steps): ¹H NMR (DMF-d₆) δ 8.68 (app s, 1H, ArH), 8.24 (d, J=1.65 Hz, 1H, H-5), 8.14-8.11 (m, 1H, ArH), 8.00-7.98 (m, 2H, ArH), 7.70-7.57 (in, 4H, H-7, ArH), 7.45 (br s, 2H, NH₂), 7.27 (d, J=8.5 8 Hz, 1 H, H-8), 6.11 (br s, 2H, NH₂), 5.41 (s, 2H, CH₂); ¹³C NMR (DMF-d₆) δ 166.73, 163.80, 162.90, 153.88, 136.22, 133.63, 133.20, 131.46, 130.08, 129.27, 129.09, 12, 128.14, 127.67, 125.75, 125.66, 124.62, 1-11.00, 67.55. Anal. (C₂₀H₁₆N₄O₂ .0.H₂O) C, H, N.

### 1:8

### (2,4-Diaminoquinazoline-6-yl)methyl diphenylacetate (103)

Compound 103 was prepared as described for 101 using diphenylacetic acid (335 mg, 1.58 mmol). The reaction was allowed to proceed for 2 days before it was filtered and worked-up as described above for 101 yielding 72 mg (36% over two steps): ¹H NMR (DMF-d₆) δ 8.08 (d, J=1.65 Hz, 1H, H-5), 7.45 (dd, J=8.58, 1.98 Hz, 1H, H-7), 7.41 (br s, 2H, NH₂), 7.37-7.19 (in, 10H, ArH), 7.19 (d, J=8.58 Hz, 1H, H-8), 6.16 (br s, 2H, NH₂), 5.25 (s, 1H, CH), 5.17 (s, 2H, CH₂); ¹³C NMR (DMF-d₆) δ 172.79, 163.73, 153.51, 140.07 (2C), 133.71, 129.25 (2C), 129.18 (2C), 128.21, 127.78 (2C), 125.44 (2C), 124.87, 110.78, 67.55, 56.93 (2C). Anal. (C₂₃H₂₀N₄O₂) C, H, N.

### 1:9

### (2,4-Diaminoquinazoline-6-yl)methyl 2-Thiophenecarboxylate (104)

Compound 104 was prepared as described for 101 using 2-thiophenecarboxylic acid (202 mg, 1.58 mmol). The reaction was allowed to proceed for 2 days before it was filtered and worked-up as described above for 101 yielding 63 mg (40% over two steps): ¹H NMR (DMF-d₆) δ 8.28 (d, J=1.65 Hz, 1H, H-5), 7.98 (br s, 2H, NH₂), 7.96 (m, 1H, ArH), 7.85-7.83 (m, 1H, ArH), 7.72 (dd, J=8.58, 1.98 Hz, 1H, H-7), 7.3 6 (d, J=8.5 8 Hz, 1 H, H-8), 7.24-7.21 (m, 1 H, ArH), 6.81 (br s, 2H, NH₂), 5.3 5 (s, 2H, CH₂); ¹³C NMR (DMF-d₆) δ 163.99, 160.37, 149.38, 134.54 (2C), 129.85, 128.99, 125.03, 123.15, 110.71, 67.21. Anal. (C₁₄H₁₂N₄O₂S. 0.5H₂O) C, H, N.

### 1:10

### (2,4-Diaminoquinazoline-6-yl)methyl 3-Thiophenecarboxylate (105)

Compound 105 was prepared using the Quest 210 organic syntheziser starting with 2,4-diamino-6-bromomethylquinazoline (20a) (0.53 mmol), 3-thiophenecarboxylic acid (202 mg, 1.58 mmol), and potassium carbonate (218 mg, 1.58 mmol) in DMF (total amount: 5 ml). The reaction was allowed to proceed for 2 days before it was filtered and rinsed with DMF. A small portion of silica gel was added to the reaction mixture and the solvent was evaporated using SpeedVac®. The silica plug was loaded on the top of a silica column, previously conditioned with dichloromethane, and the crude ester was purified as described for compound 101 yielding 34 mg (22% over two steps); ¹H NMR (DMSO-d₆) δ 8.40-8.38 (m, IH, ArH), 8.06 (d, J=1.65 Hz, 1H, H-5), 7.6:77.64 (m, 1 H, ArH), 7.56 (dd, J 8.58, 1.98 Hz, 1H, H-7), 7.50-7.48 (m, 1H, ArH), 7.3 1 (br s, 2H, NH₂), 7.20 (d, J 8.5 8 Hz, 1 H, H-8), 6.05 (hr s, 2H, NH₂), 5.26 (s, 2H, CH₂); ¹³C NMR (DMSO-d6) δ 162.44, 161.97, 161.06, 152.58, 134.09, 132.92, 132.80, 127.73, 127.49, 127.15, 124.51, 123.90, 109.89, 66.19. Anal. (C₁₄H₁₂N₄O₂S) C, H, N.

### 1:11

### (2,4-Diaminoquinazoline-6-yl)methyl 2-furoate (106)

Compound 106 was prepared as described for 105 using the Quest 210 organic syntheziser starting with 2-furoic acid (177 mg, 1.58 mmol). The reaction was allowed to proceed for 2 days before it was filtered and worked-up as described above for 105 yielding 25 mg (17% over two steps): ¹H NMR (DMSO-d₆) δ 8.40-8.38 (m, 1 H, ArH), 8.06 (d, J=1.65 Hz, 1JJ, H-5), 7.67-7.64 (m, 1H, ArH), 7.56 (dd, J=8.58, 1 98 Hz, 1H, H-7), 7.50-7.48 (m, 1H, ArH), 7.31 (br s, M, NH₂), 7.20 (d, J=8.58 Hz, 1H, H-8), 6.05 (br s, 2H, NH₂), 5.26 (s, 2H, CH₂); ¹³C NMR (DMSO-d₆) δ 162.44, 161.97, 161.06, 152.5 S, 134.09, 132.92, 132.80, 127.73, 127.49, 127.15, 124.51, 123.90, 109.89, 66.19. Anal. (C₁₄H₁₂N₄O₃.0.25H₂O) C, H, N.

### 1:12

### (2,4-Diaminoquinazoline-6-yl)methyl 3-furoate (107)

Compound 107 was prepared as described for 101 using 2-furoic acid (177 mg, 1.58 mmol). The reaction was allowed to proceed for 2 days before it was filtered and worked-up as described above for 101 yielding 30 mg (20% over two steps): ¹H NMR (DMSO-d₆) δ 8.05 (d, J=1.65 Hz, 1H, H-5), 7.96-7.95 (m, 1H, ArH), 7.56 (dd, J=8.58, 1.65 Hz, IH, H-7), 7.35-7.34 (m, 3H, NH₂+ArH), 7.20 (d, J=8.58 Hz, 1H, H-8), 6.69-6.67 (m, 1H, ArH), 6.06 (br s, 2H, NH₂), 5.27 (s, 2H, CH₂); ¹³C NMR (DMSO-d₆) δ 162.34, 160.81, 157.74, 152.19, 147.61, 143.58, 133.11, 126.84, 124.27, 124.14, 118.61, 112.28, 109.71, 66.22. Anal. (C₁₄H₁₂N₄O₃.0.75H₂O) C, H, N.

### 1:13

### (2,4-Diaminoquinazoline-6-yl)methyl 2-phenylbenzoate (108)

Compound 108 was prepared as described for 105 using the Quest 210 organic syntheziser starting with 2-phenylbenzoic acid (313 mg, 1.58 mmol). The reaction was allowed to proceed for 2 days before it was worked-up as described above for 105 yielding 47 mg (24% over two steps): ¹H NMR (DMSO-d₆) δ 7.90 (app s, 1H, H-5), 7.78-7.09 (m, 13H, ArH), 6.04 (br s, 2H, NH₂), 5.05 (s, 2H, CH₂); ¹³C NMR (DMSO-d₆) δ 168.00, 162.39, 162.11. 161.68, 161.05, 152.55, 141.23, 140.35, 132.87, 131.45, 130.93, 130.48, 129.28, 128.38, 128.13, 127.43, 127.20, 126.46, 124.32, 124.09, 109.70, 66.81. Anal. (C₂₂H₁₈N₄O₂ .0.25H₂O) C, H, N.

### 1:14

### (2,4-Diaminoquinazoline-6-yl)methyl 4-phenylbenzoate (109)

Compound 109 was prepared as described for 105 using the Quest 210 organic syntheziser starting with 4-phenylbenzoic acid (313 mg, 1.58 mmol). The reaction was allowed to proceed for 2 days before it was worked-up as described above for 105 yielding 25 mg (13% over two steps): ¹H NNM (DMSO-d₆) δ 8. 11-7.21 (m, 14H, ArH), 6.12 (br s, 2H, NH₂), 5.34 (s, 2H, CH₂); ¹³C NMR (DMSO-d₆) δ 165.48, 162.46, 160.81, 152.09, 144.76, 138.80, 133.03, 129.94, 129.11, 128.46, 128.21, 128.14, 127.30, 126.99, 124.28, 124.03, 109.89, 66.62. Anal. (C₂₂H₁₈N₄O₂ 0.75H₂O) C, H, N.

### 1:15

### (2,4-Diaminoquinazoline-6-yl)methyl 2-benzylbenzoate (110)

Compound 110 was prepared as described for 105 using the Quest 210 organic syntheziser starting with 2-benzylbenzoic acid (334 mg, 1.58 mmol). The reaction was allowed to proceed for 2 days before it was filtered and worked-up as described above for 105 yielding 53 mg (26% over two steps): ¹H NMR(DMSO-d₆) δ 8.11-7.21 (m, 14H, ArH), 6.12 (br s, 2H, NH₂), 5.34 (s, 2H, CH₂); ¹³C NMR (DMSO-d₆) δ 165.50, 162.46, 160.82, 152.09, 144.78, 138.80, 129.96, 129.11, 128.46, 128.21, 128.14, 127.30, 126.99, 124.28, 124.03, 109.89, 66.62. Anal. (C₂₃H₂₀N₄O₂) C, H, N.

### 1:16

### (2,4-Diaminoquinazoline-6-yl)methyl 3,5-dimethylbenzoate (111)

Compound 111 was prepared as described for 105 using the Quest 210 organic syntheziser starting with 3,5-dimethylbenzoic acid (237 mg, 1.58 mmol). The reaction was allowed to proceed for 2 days before it was filtered and worked-up as described above for 105 yielding 32 mg (19% over two steps). ¹H NMR (DMSO-d₆) δ 8.08 (d, J=1.65 Hz, IH, H-5), 7.32 (br s, 2H, NH₂), 7.20 (d, J=8.58 Hz, 1H, H-8), 7.28 (s, IH, ArH), 7.59-7.56 (m, 3H, NH₂+ArH), 6.06 (br s, 211, NH₂), 5.29 (s, 2H, CH₂), 3.36 (s, 6H); ¹³C NMR (DMSO-d₆) δ 165.87, 162.44, 161.09, 152.63, 138.02 (2C), 134.74, 133.08, 129.61, 127.10, 126.85 (2C), 124.57, 124.19, 109.90, 66.58, 20.68 (2C). Anal. (C₁₈H₁₈N₄O₂.1.75H₂O) C, H; N: calcd, 16.12; found, 15.50.

### 1:17

### (2,4-Diaminoquinazoline-6-yl)methyl 9-Phenanthrenecarboxylate (112)

Compound 112 was prepared as described for 105 using the Quest 210 organic syntheziser starting with 9-phenanthrenecarboxylic acid. The reaction was allowed to proceed for 2 days before it was filtered and worked-up as described above for 105 yielding 61 mg (29% over two steps): ¹H NMR (DMSO-d₆) δ 8.95-8.87 (m, 3H, ArH), 8.54 (s, 1H, ArH), 8.19-8.16 (m, 3H, ArH), 7.56-7.68 (m, 4H, H-7+ArH), 7.35 (br s, 2H, NH₂), 7.25 (d, J=8.58 Hz, 1H, H-8), 6.07 (br s, 2H, NH₂), 5.46 (s, 2H, CH₂); ¹³C NMR (DMSO-d₆) δ 166.84, 162.50, 161.13, 152.67, 133.23, 131.72, 131.40, 131.21, 130.05, 129.54, 129.45, 128.18, 127.60, 127.51, 127.35, 127.08, 125.99, 124.60, 124.32, 123.47, 123.00, 109.98, 67.07. Anal. (C₂₄H₁₈N₄O₂.1H₂O) C, H, N.

### 1:18

### (2,4-Diaminoquinazoline-6-yl)methyl 9-anthracenoate (113)

Compound 113 was prepared as described for 105 using the Quest 210 organic syntheziser starting with 9-anthracenecarboxylic acid (351 mg, 1.58 mmol). The reaction was allowed to proceed for 2 days before it was filtered and worked-up as described above for 105 yielding 50 mg (24% over two steps): ¹H NMR (DMSO-d₆) δ 8.95-8.87 (m, 3H, ArH), 8.54 (s, 1H, ArH), 8.19-8.16 (m, 3H, ArH), 7.56-7.68 (m, 4H, H-7+ArH), 7.3 5 (br s, 2H, NH₂), 7,25 (d, J=8.5 8 Hz, 1 H, H-8), 6.07 (br s, 2H, NH₂), 5.46 (s, 2H, CH₂); ¹³C NMR (DMSO-d₆) δ 168.62, 162.47, 161.12, 152:70, 133.56, 130.40, 129.21, 128.67 (2C), 127.42 (3C), 126.61, 125.78 (2C), 124.94, 124,54, 124.43 (2C), 109.90, 67.7 8 Anal. (C₂₄H₁₈N₄O₂. 0.25H₂O) C, H, N.

### 1:19

### (2,4-Diaminoquinazoline-6-yl)methyl 3-iodobenzoate (114)

Compound 114 was prepared as described for 101 starting with 2,4 diamino-6-bromomethylquinazoline (2.52 mmol), 3-iodobenzoic acid (1.87 g, 7.57 mmol), and potassium carbonate (1.04 g, 7.57 mmol) in DMF (total amount: 20 ml). The reaction was allowed to proceed for 2 days before it was filtered and worked-up as described above for 101 yielding 441 mg (42% over two steps). Recrystallization from methanol afforded 360 mg (34%) as white needles: ¹H NMR (DMSO-d₆) δ 8.25 (dd, J=1.32, 1.3 2 Hz, 1 H, ArH), 8.08 (d, J=1.65 Hz, 1 H, H-5), 8.04-7.98 (m, 2H, ArH), 7.5 9 (dd, J=8.5 8, 1.65 Hz, 1 H, H-7), 7.3 2 (br s, 2H, NH₂), 7.34 (dd, J=7.92, 7.92 Hz, 1 H, ArH), 7.20 (d, J=8.58 Hz, 1H, H-8), 6.05 (br s, 2H, NH₂), 5.32 (s, 2H, CH2); ¹³C NMR (DMSO-d₆) δ 164.00, 162.12, 160.77, 152.34, 141.56, 137.03, 132.65, 131.34, 130.64, 128.28, 126.49, 124.22, 123.76, 109.58, 94.56, 66.69. Anal. (C₁₆H₁₃IN₄O₂) C, H, N.

### 1:20

### (2,4-Diaminoquinazoline-6-yl)methyl 3-phenylbenzoate (115)

A heavy-walled glass Smith Process Vial sealed with aluminum crimp caps fitted with a silicon septum was charged with compound 114 (50 mg, 0.12 mmol) in DMF (800 μl), phenylboronic acid (72.5 mg, 0.59 mmol) in DMF (200 μL), PdCl₂(PPh₃)2 (0.8 mg, 1.2 mmol), and 2M Na₂CO₃ (150 μL), in 2 ml of DME:H₂O: EtOH (7:3:2). The reaction mixture was exposed to microwave irradiation for 120 s at 150° C. The mixture was filtered through a syringe equipped with a Titan® filter (pore size 0.45 μm). A small portion of silica gel was added to the solution. The solvent was evaporated using SpeedVac® and further purifications were conducted using flash chromatography as described for compound 101 finally yielding 15 mg (34% over two steps) of the target molecule: ¹H NMR (DMSO-d₆) δ 8.20-8.18 (m, 1H, ArH), 8.10 (d, J=1.65 Hz, 1H, H-5), 8.00-7.93 (m, 2H, ArH), 7.70-7.37 (m, 9H, ArH+NH₂), 7.22 (d, J=8.5 8 Hz, 1 H, H-8), 6.07 (br s, 2H, NH₂), 5.3 6 (s, 2H, CH₂); ¹³C NMR (DMSO-d₆) δ 165.98, 162.82, 161.22, 152.54, 141.08, 139.41, 133.46, 132.04, 132.73, 129.94, 129.49 (2C), 128.61, 128.38, 127.68, 127.55, 127.19 (2C), 124.71, 124.37, 110.21, 67.09. Anal. (C₂₂H₁₈N₄O₂. 0.75H₂O) C, H, N.

### 1:21

### (2,4-Diaminoquinazoline-6-yl)methyl[2,6-dimethoxy]-3-phenylbenzoate (116)

Compound 116 was performed as in the case of 115 using 2,6-dimethoxyphenylboronic acid (108 mg, 0.59 mmol). Work-up was conducted as described for the synthesis of 115 providing 20 mg (39% over two steps) of the target molecule: ¹H NMR (DMSO-d₆) δ 8.09 (app s, 1H, H-5), 7.92-7.89 (m, 1H, ArH), 7.78 (m, 1 H, ArH), 7.60 (dd, J=8.58, 1.98 Hz, 1H, H-7), 7.55-7.46 (m, 2H, ArH), 7.357.3 0 (m, 3 H, ArH+NH₂), 7.22 (d, J=8.5 8 Hz, 1 H, H-8), 6.7 5 (d, J=8.5 8 Hz, 1 H, H-8), 6.06 (br s, 2H, NH₂), 5.3 2 (s, 2H, CH₂) 3.64 (s, 6H, CH₃); ¹³C NMR (DMSO-d₆) δ 166.11, 162.80, 161.20, 157.32, 152.52, 136.21, 135.08, 133.50, 131.77, 129.89, 129.54, 128.54, 127.85, 127.71, 124.67, 124.46, 117.61, 110.19, 104.69, 66.93, 56.04 (2C). Anal. (C₂₄H₂₂N₄O₄.0.25H₂O) C, H, N.

The novel compounds 1:22-1:25 were prepared in an analogous manner:

| | |
|---|---|
| 1:22 | 4-Trifluoromethyl-benzoic acid 2,4- |
| | diamino-quinazoolin-6-ylmethyl ester |
| 1:23 | 2,4,5-Trifluoro-benzoic acid 2,4-diamino- |
| | quinazolin-6-ylmethyl ester |
| 1:24 | 3,5-Bis-trifluoromethyl-benzoic acid 2,4- |
| | diamino-quinazolin-6-ylmethyl ester |
| 1:25 | 4-Hexyloxy-benzoic acid 2,4-diamino- |
| | quinazolin-6-ylmethyl ester |

General procedure for the biological testing: Dextran sodium sulphate-induced colitis in mice

The Dextran sodium sulphate (DSS) model is considered to be a relevant model to study mechanisms involved in IBD in humans. Today there are several hundred articles published on the model and its response to different drugs. The immunomodulatory drug cyclosporin, given therapeutically, reduce disease activity in the DSS model in mice [Murthy S N S et al. 1993]. The DSS moodel has been evaluated by others [Cooper H S et al. 1993].

Colonic inflammation is induced by oral administration of DSS in the drinking water. An induction period of 7-10 days with DSS is followed by a treatment period of 5-10 days where DSS administration is continued and drugs or control substances are given. Parameters recorded at autopsy are body weight, spleen weight, diarrhea (wet/dry fecal weight), colon length and the histopathological appearance of the colonic tissue.

Cooper H S, Murthy S N S, Shah R S, Sedergran D J. Clinicopathologic study of dextran sulfate sodium experimental murine colitis. Lab Invest 1993;69(2):238-249.

Murthy S N S, Cooper H S. Shim H, Shah R S, Ibrahim S A, Sedergran D J. Treatment of dextran sulfate sodium-induced murine colitis by intracolonic cyclosporin. Dig Dis Sci 1993;38(9): 1722-1734.

1. A compound of formula I; 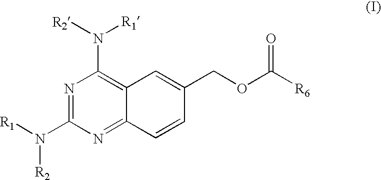
wherein R₁, R₂, R₁′ and R₂′ are H,
and either R₆ is an optionally substituted heteroaryl group selected from a mono-, bi- or tricyclic ring system comprising 1-4 heteroatoms (preferred monocyclic rings being furyl, thienyl, pyrrolyl, pyrazolyl, imidazolyl, pyridyl, pyrazinyl, pyrimidinyl, pyridazinyl, oxazolyl, isoxazolyl, thiazolyl, isothiazolyl, and the like; preferred bicyclic rings being one of the above-mentioned monocyclic rings fused to a phenyl ring such as quinolinyl, isoquinolinyl, benzimidazolyl, benzothiazolyl, benzoxazolyl, benzothienyl, indolyl, isoindolinyl and the like, or phenyl fused to an unsaturated or saturated ring such as pyranyl, pyrrolinyl, pyrrolidinyl, pyrazinyl, piperazinyl, oxazolidinyl, isooxazidinyl, morpholinyl, thiazolidinyl, and the like; and preferred tricyclic rings being acridine, xanthene, thioxanthene, phenothiazine, thianthrene, dibenzazepine, carbazol and the like),
or R₆ is an optionally substituted aryl group selected froma bicyclic or tricyclic carbocyclic, aromatic ring system (preferred systems being indanyl, naphthyl, diphenylmethyl, fluorene, anthracene, phenanthrene and the like) or a substituted phenyl ring as indicated below, 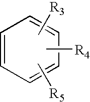
wherein R₃ is halo, C1-C6 alkyl, C1-C6 alkoxy, C1-C6 alkenyl, C1-C6 alkynyl, C1-C6 alkanoyl, thioC1-C6 alkyl, haloC1-C6 alkyl, optionally substituted phenyl, optionally substituted benzyl, furyl, optionally substituted thienyl, pyrazolyl, oxazolyl, isoxazolyl, pyrrolyl, thiazolyl or isothiazolyl,
and R₄ and R₅ are each independently either hydrogen or a substituent as defined above for R₃,
wherein, if present, the optional substituents arehalo, C₁-C₆ alkyl, C₁-C₆ alkoxy, C₁-C₆ alkenyl, C₁-C₆ alkynyl, C₁-C₆ alkanoyl, thioC₁-C₆ alkyl, haloC₁-C₆ alkyl, phenyl, benzyl, furyl, thienyl, pyrazolyl, oxazolyl, isoxazolyl, pyrrolyl, thiazolyl, isothiazolyl, NHC₁-C₆ alkyl, N(C₁-C₆alkyl)₂, thioC₁-C₆alkoxy, hydroxyC₁-C₆alkyl, aminoC₁-C₆alkyl, cyano, carbalkoxy, benzyloxy, morpholyl-C₁-C₆ alkyloxy, a monocyclic carbo- or heterocycle, as defined above, or a carbo- or heterocyclic group spaced by alkyl, such as C₁₋₃ alkylaryl.
or a pharmaceutically acceptable salt thereof.

2. A compound as claimed in claim 1, wherein R₆ is selected from; 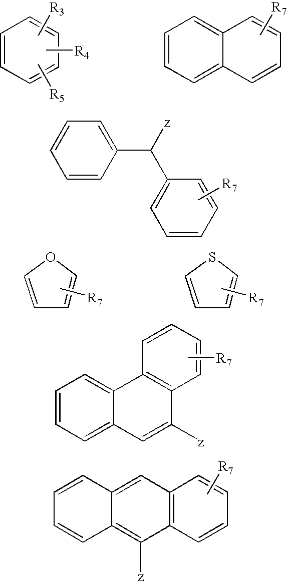
wherein z denotes the point of attachment,
R₃, R₄ and R₅ are as defined in claim 1,
and R₇ is hydrogen, halo, C₁-C₆ alkyl, C₁-C₆ alkoxy, C₁-C₆ alkenyl, C₁-C₆ alkynyl, C₁-C₆ alkanoyl, thioC₁-C₆ alkyl, haloC₁-C₆ alkyl, phenyl, benzyl, furyl, thienyl, pyrazolyl, oxazolyl, isoxazolyl, pyrrolyl, thiazolyl, isothiazolyl, NHC₁-C₆ alkyl, N(C₁-C₆ alkyl)₂, thioC₁-C₆alkoxy, hydroxyC₁-C₆alkyl, aminoC₁-C₆alkyl, cyano, carbalkoxy, benzyloxy, morpholyl-C₁-C₆ alkyloxy, a monocyclic carbo- or heterocycle, or a carbo- or heterocyclic group spaced by alkyl, such as C₁₋₃ alkylaryl.

3. A compound according to claim 2 wherein R₇ is hydrogen.

4. A compound according to any preceding claim, wherein R₃ is selected from the group consisting of halo, C₁-C₆ alkyl, haloC₁-C₆ alkyl, C₁-C₆ alkoxy, thioC₁-C₆ alkyl, phenyl, thienyl or benzyl, any of which cyclic substituents being optionally substituted as defined herein for other substituents.

5. A compound according to claim 4 wherein R₃ is selected from the group consisting of methyl, phenyl, benzyl, methoxy, n-hexoxy, iodo, fluoro, or trifluoromethyl.

6. A compound according to any previous claim wherein R₄ and R₅ are the same substituent as R₃.

7. A compound according to any previous claim wherein either R₄ or R₅ or both are not hydrogen.

8. A compound of formula I as claimed in claim 1, wherein, when R₆ represents 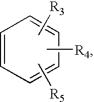
at least one hydrogen atom is in an ortho-position relative to the carbonyl group.

9. A compound according to claim 1 having one of the following formulae;

| No. | Name |
|---|---|
| 1:01 | 3,4,5-Trimethoxy-benzoic acid 2,4-diamino- |
| | quinazolin-6-ylmethyl ester |
| 1:02 | 2,5-Dimethoxy-benzoic acid 2,4-diamino- |
| | quinazolin-6-ylmethyl ester |
| 1:03 | 2,3,4-Trimethoxy-benzoic acid 2,4-diamino- |
| | quinazolin-6-ylmethyl ester |
| 1:04 | 3,5-Dimethoxy-benzoic acid 2,4-diamino- |
| | quinazolin-6-ylmethyl ester |
| 1:05 | 3,4-Dimethoxy-benzoic acid 2,4-diamino- |
| | quinazolin-6-ylmethyl ester |
| 1:06 | (2,4-Diaminoquinazoline-6-yl)methyl 1- |
| | Naphthoate |
| 1:07 | (2,4-Diaminoquinazoline-6-yl)methyl 2- |
| | Naphthoate |
| 1:08 | (2,4-Diaminoquinazoline-6-yl)methyl |
| | diphenylacetate |
| 1:09 | (2,4-Diaminoquinazoline-6-yl)methyl 2- |
| | Thiophenecarboxylate |
| 1:10 | (2,4-Diaminoquinazoline-6-yl)methyl 3- |
| | Thiophenecarboxylate |
| 1:11 | (2,4-Diaminoquinazoline-6-yl)methyl 2-furoate |
| 1:12 | (2,4-Diaminoquinazoline-6-yl)methyl 3-furoate |
| 1:13 | (2,4-Diaminoquinazoline-6-yl)methyl 2- |
| | phenylbenzoate |
| 1:14 | (2,4-Diaminoquinazoline-6-yl)methyl 4- |
| | phenylbenzoate |
| 1:15 | (2,4-Diaminoquinazoline-6-yl)methyl 2- |
| | benzylbenzoate |
| 1:16 | (2,4-Diaminoquinazoline-6-yl)methyl 3,5- |
| | dimethylbenzoate |
| 1:17 | (2,4-Diaminoquinazoline-6-yl)methyl 9- |
| | Phenanthrenecarboxylate |
| 1:18 | (2,4-Diaminoquinazoline-6-yl)methyl 9- |
| | anthracenoate |
| 1:19 | (2,4-Diaminoquinazoline-6-yl)methyl 3- |
| | iodobenzoate |
| 1:20 | (2,4-Diaminoquinazoline-6-yl)methyl 3- |
| | phenylbenzoate |
| 1:21 | (2,4-Diaminoquinazoline-6-yl)methyl[2,6- |
| | dimethoxy]-3-phenylbenzoate |
| 1:22 | 4-Trifluoromethyl-benzoic acid 2,4-diamino- |
| | quinazolin-6-ylmethyl ester |
| 1:23 | 2,4,5-Trifluoro-benzoic acid 2,4-diamino- |
| | quinazolin-6-ylmethyl ester |
| 1:24 | 3,5-Bis-trifluoromethyl-benzoic acid 2,4-diamino- |
| | quinazolin-6-ylmethyl ester |
| 1:25 | 4-Hexyloxy-benzoic acid 2,4-diamino-quinazolin- |
| | 6-ylmethyl ester |

10. A process for the production of a compound as claimed in claim 1 comprising the reaction of a compound of Formula II, protected or activated as necessary, with a compound of Formula III, protected or activated as necessary, followed by deprotection and/or salt formation where necessary or desired, 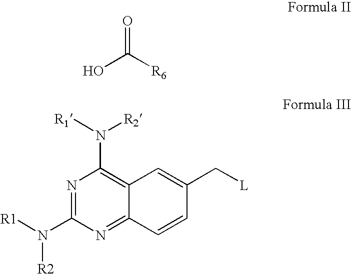
wherein R₆, R₁, R₂, R₁′ and R₂′ are as defined in claim 1 and L is a suitable leaving group.

11. A process for the production of a compound as claimed in claim 1 comprising the reaction of a compound of Formula IV, protected or activated as necessary, with a compound of Formula V, protected or activated as necessary, followed by deprotection and/or salt formation where necessary or desired, 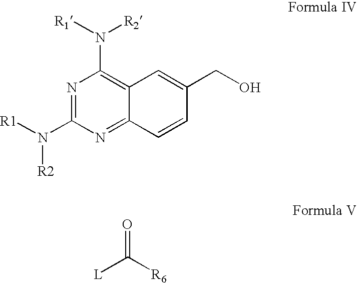
wherein R₆, R₁, R₂, R₁′ and R₂′ are as defined in claim 1 and L is a suitable leaving group.

12. A pharmaceutical composition comprising a compound as defined in any preceding claim and a pharmaceutically acceptable carrier or diluent therefor.

13. A compound as defined in any of claims 1-9 for use in therapy.

14. Use of a compound as defined in any of claims 1-9 in the manufacture of a medicament for the treatment of diseases or conditions which can be therapeutically treated by immuno-modulating or cytostatic compounds, in particular dihydrofolate reductase inhibitors, either applied topically, orally, rectally, or parenterally, or cancer forms being sensitive to methotrexate, inflammatory bowel disease i.e. ulcerative colitis and Crohn's disease, asthma, other serious pulmonary diseases, Pneumocystis carinii pneumonia (PCP), psoriasis, inflammations caused by bacteria, fungi, protozoa, rheumatoid arthritis as well as other inflammatory conditions, colorectal cancer, cancer of the urinary bladder, the skin, the lung and other cancer types that may be reached from the “outside” of the body, non-surgical abortions (intrauterin administration), or liver or intestine transplantations by preventing immunogenic rejection reactions.

15. Method for treating diseases or conditions which can be therapeutically treated by immuno-modulating or cytostatic compounds, in particular dihydrofolate reductase inhibitors, either applied topically, orally or parenterally, or cancer forms being sensitive to methotrexate, inflammatory bowel disease i.e. ulcerative colitis and Crohn's disease, colorectal cancer, asthma, or other serious pulmonary diseases Pneumocystis carinii pneumonia (PCP), psoriasis, inflammations caused by bacteria, fungi, protozoa, rheumatoid arthritis as well as other inflammatory conditions, cancer of the urinary bladder, the skin, the lung and other cancer types that are reachable by topical application, non-surgical abortions (intrauterin administration), liver and intestine transplantations by preventing immunogenic rejection reactions, whereby a therapeutically effective amount of at least one compound defined in claims 1-9 is administered for a time sufficient to substantially eliminate the signs and symptoms of such a disease

16. Use as claimed in claim 14 or method of treatment as claimed in claim 15 where the disease is a disease which is sensitive to an inhibition of dihydrofolate reductase.

17. Use as claimed in claim 14 or method of treatment as claimed in claim 15 where the disease is a cancer form sensitive to methotrexate.

18. Use as claimed in claim 14 or method of treatment as claimed in claim 15 where the disease is inflammatory bowel disease.

19. Use as claimed in claim 14 or method of treatment as claimed in claim 15 where the disease is Pneumocystis carinii pneumonia.

20. Use as claimed in claim 14 or method of treatment as claimed in claim 15 where the disease is psoriasis.

21. Use as claimed in claim 14 or method of treatment as claimed in claim 15 where the disease is rheumatoid arthritis.

22. Use as claimed in claim 14 or method of treatment as claimed in claim 15 where the disease is inflammation caused by fungal, protozoal and/or bacterial infections.

23. Use as claimed in claim 14 or method of treatment as claimed in claim 15 where the disease is asthma or a pulmonary disease.

24. Use as claimed in claim 14 or method of treatment as claimed in claim 15 for liver and intestine transplantations by preventing immunogenic rejection reactions.
